(19) United States
(12) Patent Application Publication
(10) Pub. No.: US 2018/0016619 A1
(43) Pub. Date: Jan. 18, 2018

(54) Rapid Detection of Microbial Resistance to Lactam Antibiotics by LC-MS/MS

(71) Applicant: DH Technologies Development Pte. Ltd., Singapore (SG)

(72) Inventors: Michael Jarvis, Alliston (CA); Larissa Matukas, Toronto (CA); Alex Romaschin, Toronto (CA)

(21) Appl. No.: 15/550,294

(22) PCT Filed: Feb. 9, 2016
(86) PCT No.: PCT/IB2016/050677

§ 371 Date: Aug. 10, 2017

Related U.S. Application Data
(60) Provisional application No. 62/114,852, filed on Feb. 11, 2015.

(51) Int. Cl. C12Q 1/18; G01N 30/72; G01N 30/88; H01J 49/00

(52) U.S. Cl. CPC C12Q 1/18; G01N 30/88; G01N 30/7266; G01N 2030/8813; H01J 49/0036; H01J 49/0045; G01N 2560/00

(57) ABSTRACT

A rapid screening process is provided for identification of bacterial resistance to antibiotics by utilizing LC-MS/MS to quantitate concentrations of parent drugs and also detect hydrolysis products which result from beta-lactamase activity. The susceptibility testing is accomplished in time periods as short as 90 minutes, which includes incubation of bacteria with antibiotics and LC-MS/MS analysis. The antibiotics can be multiplexed for incubation with bacteria to minimize analysis time. 23 different strains of E. coli have been evaluated by this method including ATCC reference (3) as well as clinical isolates (20) and achieved complete concordance with traditional methods. To date the following antibiotics have been tested: penicillin, ampicillin, amoxicillin, cloxacillin, piperacillin/tazobactam, and cefotaxime. All incubations are conducted in the absence and presence of tazobactam which acts as a control. LC-MS/MS analysis was conducted on an AB SCIEX 3200 QTRAP system utilizing positive ion electrospray with MRM detection.

### CROSS REFERENCE TO RELATED APPLICATION

This application claims the benefit of U.S. Provisional Patent Application Ser. No. 62/114,852, filed Feb. 11, 2015, the content of which is incorporated by reference herein in its entirety.

### INTRODUCTION

The teachings herein relate to methods and systems for determining microbial resistance to an antibiotic by detecting changes in the antibiotic rather than changes in bacterial cell growth. More particularly, bacterial resistance antibiotics is identified by utilizing liquid chromatography coupled mass spectrometry/mass spectrometry (LC-MS/MS) to quantitate concentrations of parent lactam antibiotics and also detect hydrolysis products that result from beta-lactamase activity of the bacteria.

### BACKGROUND

Matrix assisted laser desorption ionization time-of-flight (MALDI-TOF) mass spectrometry has revolutionized bacterial identification based on patterns of ribosomal protein expression. However, bacterial resistance to antibiotics is still generally determined by conventional methods that evaluate bacterial growth in the presence of antibiotics. The growth of bacterial cells is determined in a number of ways. Turbidometric methods measure the amount of light absorbed by bacterial cells to quantify their growth. Spectrophotometric methods measure the reflection or transmission properties of bacterial cells as a function of wavelength to quantify their growth. Finally, disk diffusion methods involve placing antibiotic-impregnated wafers or disks on an agar plate where bacterial cells are grown. The wafers or disks are then analyzed after a period of time to determine if bacterial cell growth is visibly inhibited around the wafers or disks.

All of these conventional methods to determine the resistance of a bacterial microbe to a specific antibiotic are relatively slow processes that often require 12-24 hours. They require a large amount of time due to incubation. Essentially, in these methods, the bacterial cells extracted need to be incubated for a large amount of time in order to provide cell growth that is large enough to be detected.

Bacterial sepsis and septic shock are major causes of mortality worldwide. In the U.S. it is estimated that 250,000 patients a year develop life threatening infections with a mortality rate that varies from 28 to greater than 50% depending upon other underlying disease conditions and the severity of infection. Unfortunately, time is of the essence in treating bacterial infections. The sooner antibiotic resistance can be determined, the more likely a patient can be successfully treated.

As a result, systems and methods are needed to determine microbial resistance to antibiotics more quickly than conventional methods that rely on detecting bacterial cell growth.

### SUMMARY

A system is disclosed for detecting the resistance of a bacterial microbe to one or more antibiotic drugs. System includes an incubation device, a separation device, an ion source device, a tandem mass spectrometer, and a processor.

The Incubation device incubates a sample mixture of a bacterial microbe and one or more antibiotic drugs over a first time period. Initial concentrations of the one or more antibiotic drugs in the sample mixture is known.

The separation device separates the one or more antibiotic drugs from the incubated mixture over a second time period that follows the first time period.

The ion source device repeatedly transforms the separating one or more antibiotic drugs into ions over the second time period.

The tandem mass spectrometer repeatedly selects and fragments the ions of the one or more antibiotic drugs over the second time period. Repeatedly selecting and fragmenting the ions of the one or more antibiotic drugs produces a plurality of product ion spectra for the one or more antibiotic drugs over the second time period.

The processor is in communication with the tandem mass spectrometer. The processor calculates a chromatogram for product ions of the one or more antibiotic drugs from the plurality of product ion spectra. The processor calculates measured concentrations of the one or more antibiotic drugs from the chromatogram. The processor compares the measured concentrations to the initial concentrations. The processor reports the detection of the resistance of the bacterial microbe to an antibiotic drug of the one or more antibiotic drugs if a measured concentration of the antibiotic drug is less than an initial concentration of the antibiotic drug by a predetermined amount.

A method is disclosed for detecting the resistance of a bacterial microbe to one or more antibiotic drugs. A sample mixture of a bacterial microbe and one or more antibiotic drugs is incubated over a first time period using an incubation device. Initial concentrations of the one or more antibiotic drugs in the sample mixture is known.

The one or more antibiotic drugs are separated from the incubated mixture over a second time period that follows the first time period using a separation device.

The separated one or more antibiotic drugs are repeatedly transformed into ions over the second time period using an ion source device.

The ions of the one or more antibiotic drugs are repeatedly selected and fragmented over the second time period using a tandem mass spectrometer. Repeatedly selecting and fragmenting the ions of the one or more antibiotic drugs produces a plurality of product ion spectra for the one or more antibiotic drugs over the second time period.

A chromatogram for product ions of the one or more antibiotic drugs is calculated from the plurality of product ion spectra using a processor.

Measured concentrations of the one or more antibiotic drugs are calculated from the chromatogram using the processor.

The measured concentrations are compared to the initial concentrations using the processor.

The detection of the resistance of the bacterial microbe to an antibiotic drug of the one or more antibiotic drugs is reported if a measured concentration of the antibiotic drug is less than an initial concentration of the antibiotic drug by a predetermined amount using the processor.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is a chromatogram showing the detection of ampicillin and piperacillin parent drugs in a sensitive strain with no hydrolysis of antibiotics, in accordance with various embodiments.

FIG. 3 is a chromatogram showing a resistant strain which hydrolyses both drugs with the appearance of hydrolyzed product, in accordance with various embodiments.

FIG. 4 is a chromatogram showing the cefotaxime parent drug in the absence of hydrolysis, in accordance with various embodiments.

FIG. 5 is a chromatogram showing the disappearance of the cefotaxime parent drug in the presence of an E. coli expressing an extended spectrum beta-lactamase.

FIG. 6 is a schematic diagram of system for detecting the resistance of a bacterial microbe to one or more antibiotic drugs, in accordance with various embodiments.

FIG. 7 is a flowchart showing a method for detecting the resistance of a bacterial microbe to one or more antibiotic drugs, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

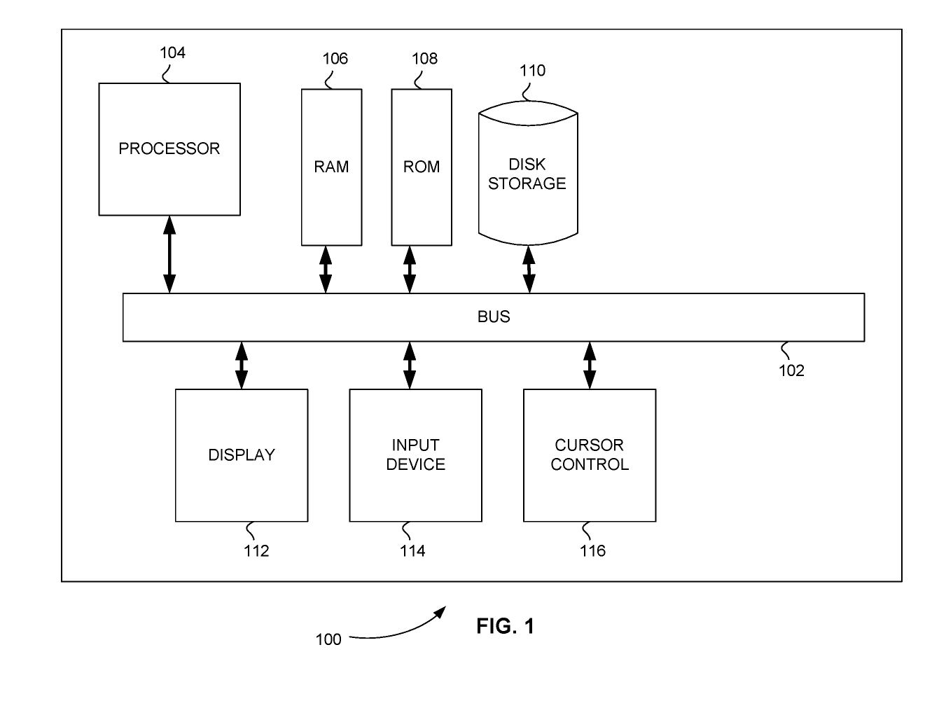

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein. Alternatively hard-wired circuitry may be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium may take many forms, including but not limited to, non-volatile media, volatile media, and transmission media. Non-volatile media includes, for example, optical or magnetic disks, such as storage device 110. Volatile media includes dynamic memory, such as memory 106. Transmission media includes coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Rapid Detection of Antibiotic Resistance to Antibiotics Using Liquid Chromatography-Tandem Mass Spectrometry (LC-MS/MS)

As described above, bacterial resistance to antibiotics is still generally determined by conventional methods that evaluate bacterial growth in the presence of antibiotics. All of these conventional methods to determine the resistance of a bacterial microbe to a specific antibiotic are relatively slow processes that often require 12-24 hours, due to incubation. Essentially, in these methods, the bacterial cells extracted need to be incubated for a large amount of time in order to provide cell growth that is large enough to be detected.

Unfortunately, time is of the essence in treating bacterial infections. The sooner antibiotic resistance can be determined, the more likely a patient can be successfully treated. As a result, systems and methods are needed to determine microbial resistance to antibiotics more quickly than conventional methods that rely on detecting bacterial cell growth.

In various embodiments, methods and systems determine microbial resistance to an antibiotic by detecting changes in the antibiotic rather than changes in bacterial cell growth. More particularly, bacterial resistance antibiotics is identified by utilizing liquid chromatography coupled mass spectrometry/mass spectrometry (LC-MS/MS) to quantitate concentrations of parent lactam antibiotics and also detect hydrolysis products that result from beta-lactamase activity of the bacteria.

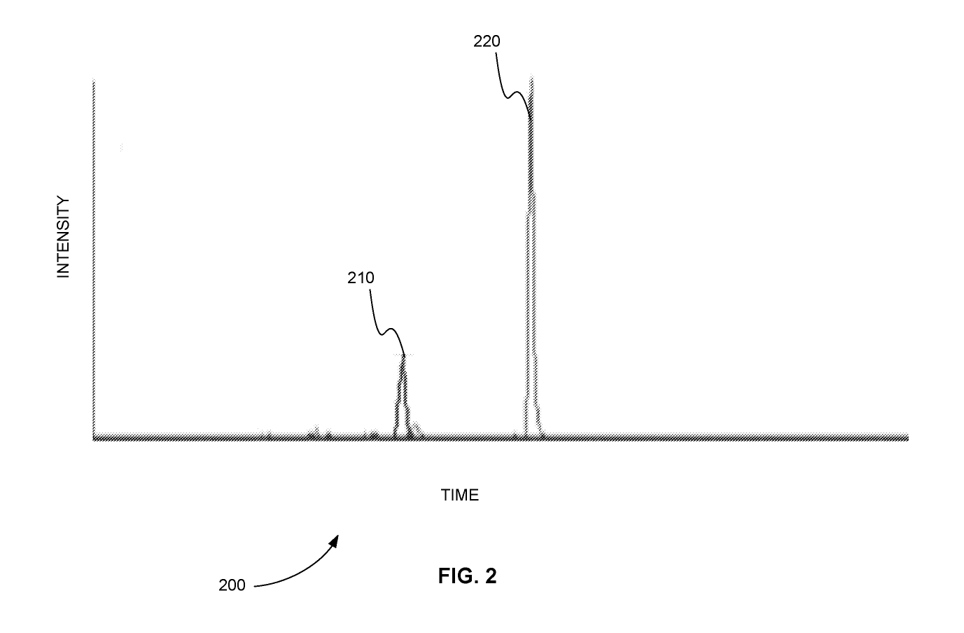

This susceptibility testing of antibiotics is accomplished in time periods as short as 90 minutes, which includes incubation of bacteria with antibiotics and LC-MS/MS analysis. In addition, multiple antibiotics can be analyzed within the same shorter time period. In other words, the antibiotics can be multiplexed for incubation with bacteria to minimize analysis time. 23 different strains of E. coli have been evaluated by this method including ATCC references (3) as well as clinical isolates (20). These evaluations achieved complete concordance with traditional methods. To date the following antibiotics have been tested: penicillin, ampicillin, amoxicillin, cloxacillin, piperacillin/tazobactam, and cefotaxime. All incubations are conducted in the absence and presence of tazobactam which acts as a control. LC-MS/MS analysis was conducted on an AB SCIEX 3200 QTRAP system utilizing positive ion electrospray with multiple reaction monitoring (MRM) detection and drug separation on a C18 reverse phase column using a linear methanol gradient. A sample chromatographic profile is shown in the following FIGS. 2-5. The data shown in these figures is found by incubating Clinical E. coli isolates with a mixture of ampicillin, piperacillin and cefotaxime and subjecting the isolates to LC-MS/MS analysis on an AB SCIEX 3200 QTRAP hybrid triple quadrupole/linear ion trap mass spectrometer.

FIG. 2 is a chromatogram 200 showing the detection of ampicillin and piperacillin parent drugs in a sensitive strain of E. coli bacteria with no hydrolysis of antibiotics, in accordance with various embodiments. Only the parent drugs ampicillin 210 and piperacillin 220 are shown in chromatogram 200.

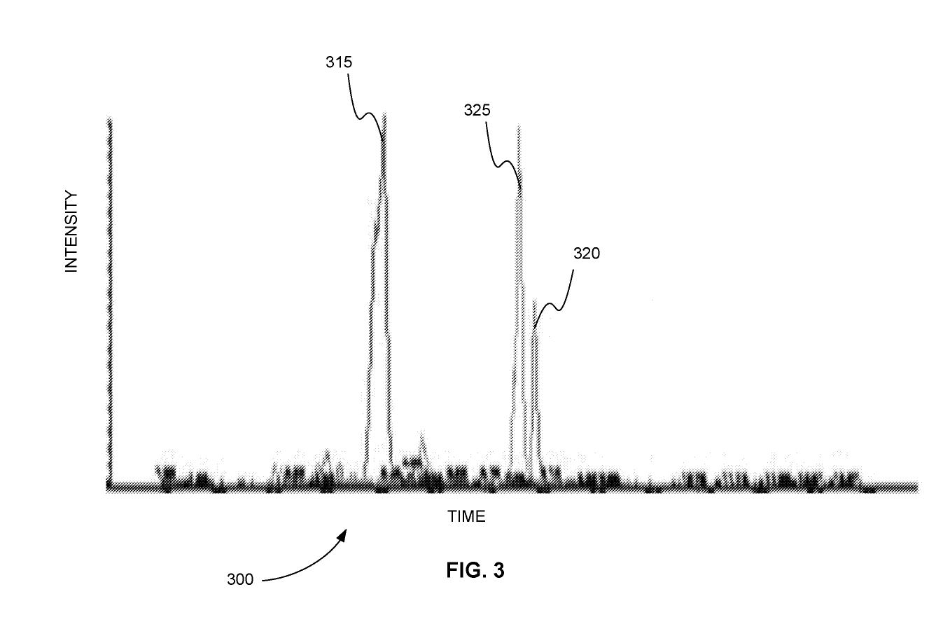

FIG. 3 is a chromatogram 300 showing the appearance of hydrolyzed products produced by a resistant strain of E. coli bacteria that hydrolyses both antibiotic drugs, in accordance with various embodiments. Ampicillin hydrolysis 315 and piperacillin hydrolysis 325 are present in chromatogram 300. The parent drug piperacillin 320 is also shown in chromatogram 300.

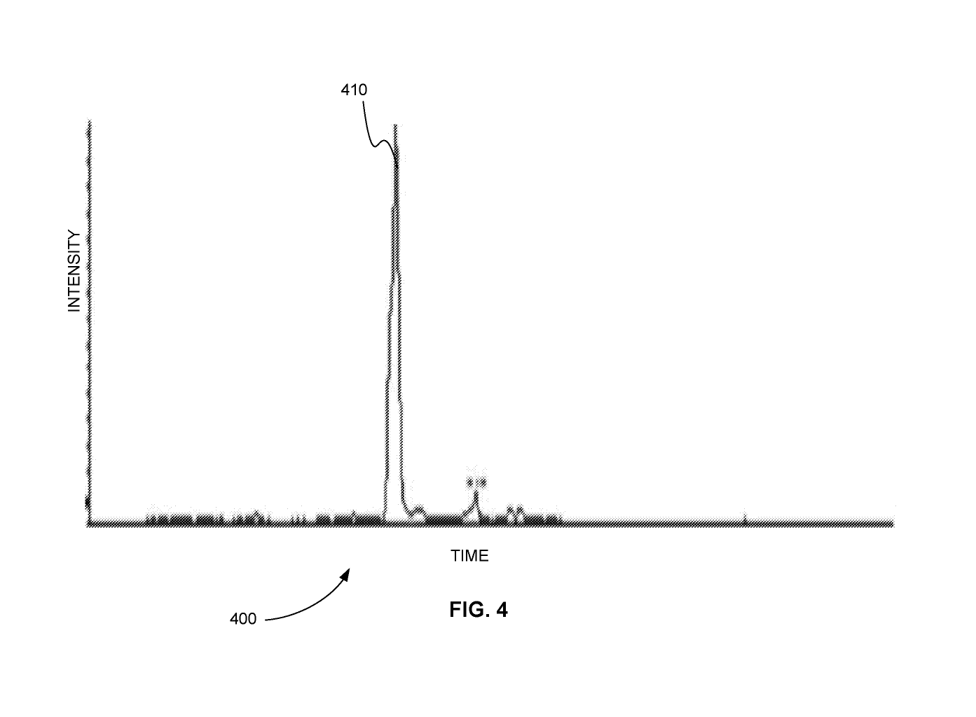

FIG. 4 is a chromatogram 400 showing the detection of the cefotaxime parent drug from a sample with a sensitive strain of E. coli bacteria, in accordance with various embodiments. Only the parent drug cefotaxime 410 is shown in chromatogram 400. There is no hydrolysis of the cefotaxime parent drug present in chromatogram 400.

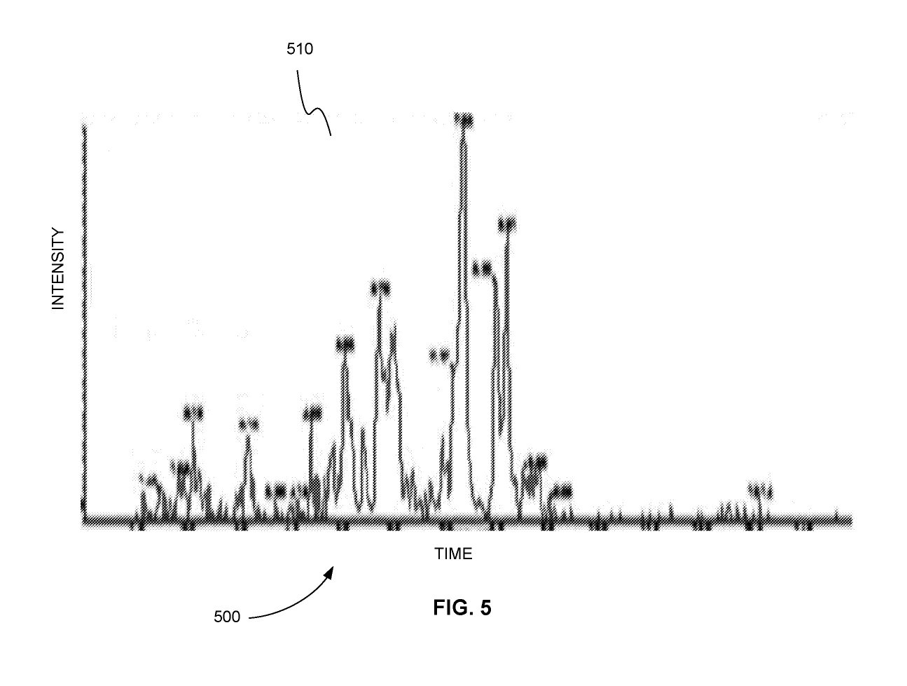

FIG. 5 is a chromatogram 500 showing the disappearance of the cefotaxime parent drug in the presence of a resistant strain of E. coli expressing an extended spectrum beta-lactamase. Note that the cefotaxime parent drug is missing at location 510. Also note that the peak intensities in chromatogram 500 of FIG. 5 are 1.2 times smaller than the peak intensities in chromatogram 400 of FIG. 4.

### System for Detecting Antibiotic Resistance

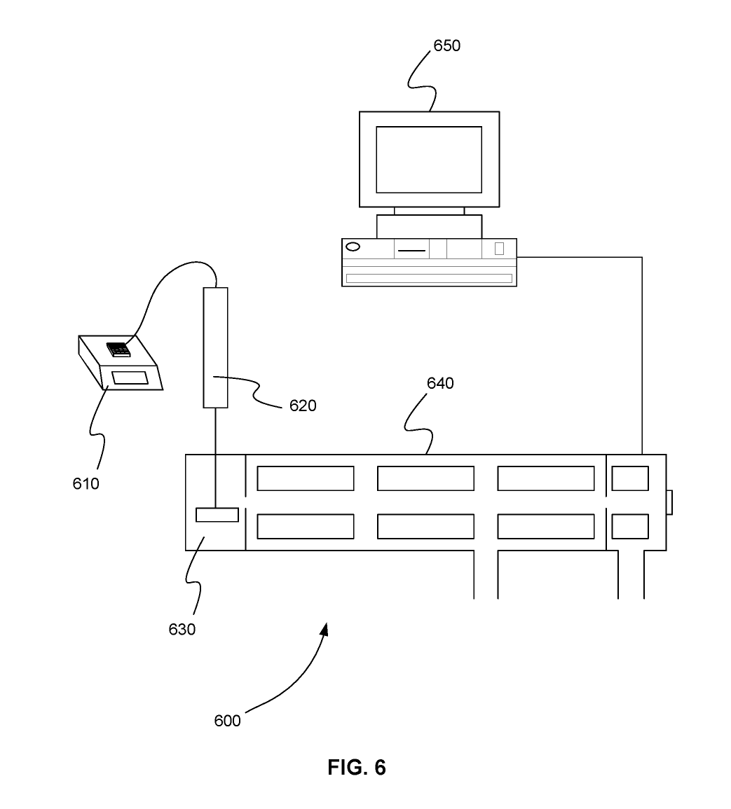

FIG. 6 is a schematic diagram 600 of system for detecting the resistance of a bacterial microbe to one or more antibiotic drugs, in accordance with various embodiments.

System 600 includes incubation device 610, separation device 620, ion source device 630, tandem mass spectrometer 640, and processor 650.

Incubation device 610 incubates a sample mixture of a bacterial microbe and one or more antibiotic drugs over a first time period. Initial concentrations of the one or more antibiotic drugs in the sample mixture is known.

Separation device 620 separates the one or more antibiotic drugs from the incubated mixture over a second time period that follows the first time period. Separation device 620 can perform a separation technique that includes, but is not limited to, liquid chromatography, gas chromatography, capillary electrophoresis, or ion mobility.

Ion source device 630 can be part of tandem mass spectrometer 640, or can be a separate device. Ion source device 630 repeatedly transforms the separating one or more antibiotic drugs into ions over the second time period.

Tandem mass spectrometer 640, for example, can include one or more physical mass filters and one or more physical mass analyzers. A mass analyzer of tandem mass spectrometer 640 can include, but is not limited to, a time-of-flight (TOF), quadrupole, an ion trap, a linear ion trap, an orbitrap, or a Fourier transform mass analyzer. Tandem mass spectrometer 640 can include separate stages or steps in space or time, respectively.

Tandem mass spectrometer 640 repeatedly selects and fragments the ions of the one or more antibiotic drugs over the second time period. Repeatedly selecting and fragmenting the ions of the one or more antibiotic drugs produces a plurality of product ion spectra for the one or more antibiotic drugs over the second time period.

Processor 650 can be, but is not limited to, a computer, microprocessor, or any device capable of sending and receiving control signals and data from tandem mass spectrometer 640 and processing data. Processor 650 can be, for example, computer system 100 of FIG. 1. In various embodiments, processor 650 is in communication with tandem mass spectrometer 640.

Processor 650 calculates a chromatogram for product ions of the one or more antibiotic drugs from the plurality of product ion spectra. Processor 650 calculates measured concentrations of the one or more antibiotic drugs from the chromatogram. Processor 650 compares the measured concentrations to the initial concentrations. Processor 650 reports the detection of the resistance of the bacterial microbe to an antibiotic drug of the one or more antibiotic drugs if a measured concentration of the antibiotic drug is less than an initial concentration of the antibiotic drug by a predetermined amount.

In various embodiments, the summation of the first time period and the second time period is less than 90 minutes.

In various embodiments, separation device 620 of FIG. 6 further separates one or more hydrolyzed components of the one or more antibiotic drugs over the second time period. Ion source device 630 further repeatedly transforms the separating one or more hydrolyzed components into ions over the second time period. Tandem mass spectrometer 640 further repeatedly selects and fragments the ions of the one or more hydrolyzed components over the second time period, producing a plurality of product ion spectra for the one or more hydrolyzed components over the second time period. Processor 650 further calculates the chromatogram for product ions of the one or more hydrolyzed components from the plurality of product ion spectra. Processor 650 further calculates measured concentrations of the one or more hydrolyzed components from the chromatogram. Processor 650 further reports the detection of the resistance of the bacterial microbe to an antibiotic drug of the one or more antibiotic drugs if a measured concentration of a hydrolyzed component of the antibiotic drug is greater than a predetermined threshold amount.

In various embodiments, the one or more antibiotic drugs comprise penicillin.

In various embodiments, the one or more antibiotic drugs comprise ampicillin.

In various embodiments, the one or more antibiotic drugs comprise amoxicillin.

In various embodiments, the one or more antibiotic drugs comprise cloxacillin.

In various embodiments, the one or more antibiotic drugs comprise piperacillin/tazobactum.

In various embodiments, the one or more antibiotic drugs comprise cefotaxime.

In various embodiments, separation device 620 comprises a C18 reverse phase chromatographic column.

In various embodiments, ion source device 630 comprises positive ion electrospray.

In various embodiments, tandem mass spectrometer 640 selects and fragments ions using MRM.

### Method for Detecting Antibiotic Resistance

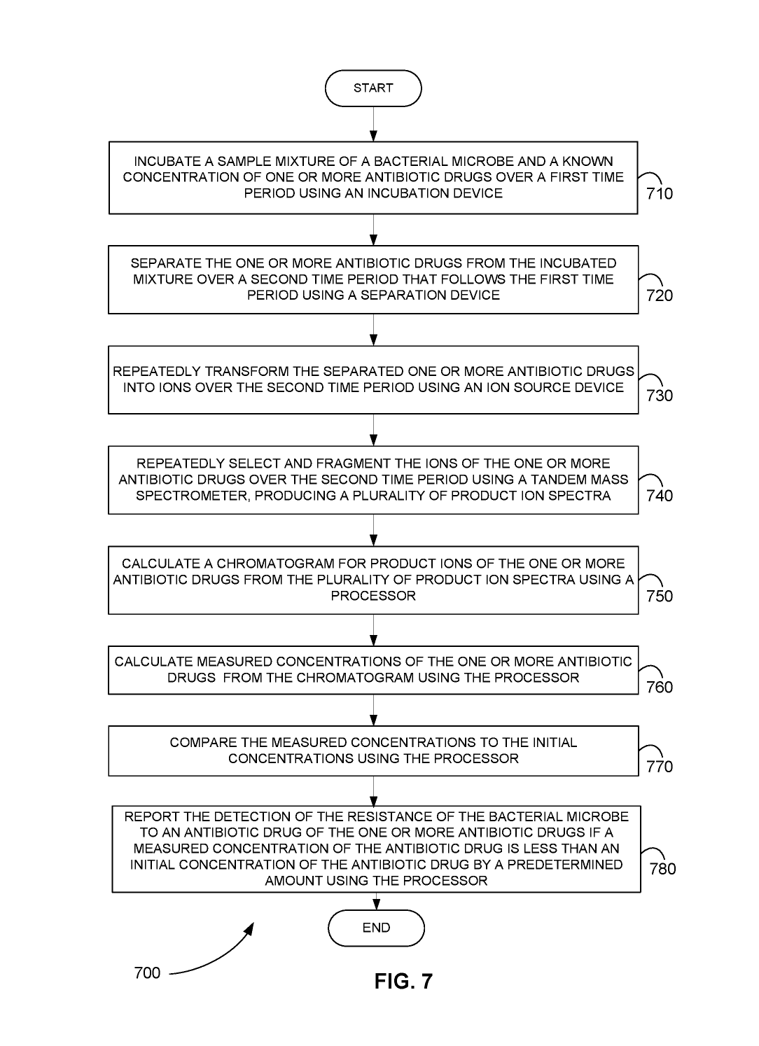

FIG. 7 is a flowchart showing a method 700 for detecting the resistance of a bacterial microbe to one or more antibiotic drugs, in accordance with various embodiments.

In step 710 of method 700, a sample mixture of a bacterial microbe and one or more antibiotic drugs is incubated over a first time period using an incubation device. Initial concentrations of the one or more antibiotic drugs in the sample mixture is known.

In step 720, the one or more antibiotic drugs are separated from the incubated mixture over a second time period that follows the first time period using a separation device.

In step 730, the separated one or more antibiotic drugs are repeatedly transformed into ions over the second time period using an ion source device.

In step 740, the ions of the one or more antibiotic drugs are repeatedly selected and fragmented over the second time period using a tandem mass spectrometer. Repeatedly selecting and fragmenting the ions of the one or more antibiotic drugs produces a plurality of product ion spectra for the one or more antibiotic drugs over the second time period.

In step 750, a chromatogram for product ions of the one or more antibiotic drugs is calculated from the plurality of product ion spectra using a processor.

In step 760, measured concentrations of the one or more antibiotic drugs are calculated from the chromatogram using the processor.

In step 770, the measured concentrations are compared to the initial concentrations using the processor.

In step 780, the detection of the resistance of the bacterial microbe to an antibiotic drug of the one or more antibiotic drugs is reported if a measured concentration of the antibiotic drug is less than an initial concentration of the antibiotic drug by a predetermined amount using the processor.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A system for detecting the resistance of a bacterial microbe to one or more antibiotic drugs, comprising:
an incubation device configured to incubate a sample mixture of a bacterial microbe and one or more antibiotic drugs over a first time period, wherein initial concentrations of the one or more antibiotic drugs in the sample mixture is known;
a separation device configured to separate the one or more antibiotic drugs from the incubated mixture over a second time period that follows the first time period;
an ion source device configured to repeatedly transform the separated one or more antibiotic drugs into ions over the second time period;
a tandem mass spectrometer configured to repeatedly select and fragment the ions of the one or more antibiotic drugs over the second time period, producing a plurality of product ion spectra for the one or more antibiotic drugs over the second time period; and
a processor in communication with the tandem mass spectrometer configured to
calculate a chromatogram for product ions of the one or more antibiotic drugs from the plurality of product ion spectra,
calculate measured concentrations of the one or more antibiotic drugs from the chromatogram,
compare the measured concentrations to the initial concentrations, and
report the detection of the resistance of the bacterial microbe to an antibiotic drug of the one or more antibiotic drugs if a measured concentration of the antibiotic drug is less than an initial concentration of the antibiotic drug by a predetermined amount.

2. The system of claim 1, wherein the summation of the first time period and the second time period is less than 90 minutes.

3. The system of claim 1, wherein
the separation device is further configured to separate one or more hydrolyzed components of the one or more antibiotic drugs over the second time period,
the ion source device is further configured to repeatedly transform the separated one or more hydrolyzed components into ions over the second time period,
the tandem mass spectrometer is further configured to repeatedly select and fragment the ions of the one or more hydrolyzed components over the second time period, producing a plurality of product ion spectra for the one or more hydrolyzed components over the second time period, and
the processor is further configured to
calculate the chromatogram for product ions of the one or more hydrolyzed components from the plurality of product ion spectra,
calculate measured concentrations of the one or more hydrolyzed components from the chromatogram, and
report the detection of the resistance of the bacterial microbe to an antibiotic drug of the one or more antibiotic drugs if a measured concentration of a hydrolyzed component of the antibiotic drug is greater than a predetermined threshold amount.

4. The system of claim 1, wherein the one or more antibiotic drugs comprise penicillin.

5. The system of claim 1, wherein the one or more antibiotic drugs comprise ampicillin.

6. The system of claim 1, wherein the one or more antibiotic drugs comprise amoxicillin.

7. The system of claim 1, wherein the one or more antibiotic drugs comprise cloxacillin.

8. The system of claim 1, wherein the one or more antibiotic drugs comprise piperacillin/tazobactum.

9. The system of claim 1, wherein the one or more antibiotic drugs comprise cefotaxime.

10. The system of claim 1, wherein the separation device comprises a C18 reverse phase chromatographic column.

11. The system of claim 1, wherein the ion source device comprises positive ion electrospray.

12. The system of claim 1, wherein the ion source device comprises positive ion electrospray.

13. The system of claim 1, wherein the tandem mass spectrometer selects and fragments ions using multiple reaction monitoring (MRM).

14. A method for detecting the resistance of a bacterial microbe to one or more antibiotic drugs, comprising:
incubating a sample mixture of a bacterial microbe and one or more antibiotic drugs over a first time period using an incubation device, wherein initial concentrations of the one or more antibiotic drugs in the sample mixture is known.
separating the one or more antibiotic drugs from the incubated mixture over a second time period that follows the first time period using a separation device;
repeatedly transforming the separated one or more antibiotic drugs into ions over the second time period using an ion source device;
repeatedly selecting and fragmenting the ions of the one or more antibiotic drugs over the second time period using a tandem mass spectrometer, producing a plurality of product ion spectra for the one or more antibiotic drugs over the second time period;
calculating a chromatogram for product ions of the one or more antibiotic drugs from the plurality of product ion spectra using a processor;
calculating measured concentrations of the one or more antibiotic drugs from the chromatogram using the processor,
comparing the measured concentrations to the initial concentrations using the processor, and
reporting the detection of the resistance of the bacterial microbe to an antibiotic drug of the one or more antibiotic drugs if a measured concentration of the antibiotic drug is less than an initial concentration of the antibiotic drug by a predetermined amount using the processor.

15. The method of claim 14, wherein
the separating step further includes separating one or more hydrolyzed components of the one or more antibiotic drugs over the second time period,
the transforming step further includes repeatedly transforming the separating one or more hydrolyzed components into ions over the second time period,
the selecting and fragmenting step further includes repeatedly selecting and fragmenting the ions of the one or more hydrolyzed components over the second time period, producing a plurality of product ion spectra for the one or more hydrolyzed components over the second time period,
the calculating the chromatogram step further includes calculating the chromatogram for product ions of the one or more hydrolyzed components from the plurality of product ion spectra, and
the reporting step further includes reporting the detection of the resistance of the bacterial microbe to an antibiotic drug of the one or more antibiotic drugs if a measured concentration of a hydrolyzed component of the antibiotic drug is greater than a predetermined threshold amount.
